(19) United States
(12) Patent Application Publication
(10) Pub. No.: US 2018/0289653 A1
(43) Pub. Date: Oct. 11, 2018

(54) LIPID FORMULATIONS CONTAINING BIOACTIVE FATTY ACIDS AND A NON-FATTY ACID ANTI-INFLAMMATORY AGENT

(71) Applicant: SCIADONICS, INC., Long Lake, MN (US)

(72) Inventor: Alvin Berger, Long Lake, MN (US)

(21) Appl. No.: 16/062,981

(22) PCT Filed: Dec. 16, 2016
(86) PCT No.: PCT/US16/67090

§ 371 Date: Jun. 15, 2018

Related U.S. Application Data
(60) Provisional application No. 62/269,494, filed on Dec. 18, 2015.

(51) Int. Cl. A61K 31/202; A61K 31/201; A61P 29/00

(52) U.S. Cl. CPC A61K 31/202; A61K 31/201; A61K 45/06; A61K 2300/00; A61P 29/00

(57) ABSTRACT

Provided herein is technology relating to compositions containing bioactive fatty acids in combination with one or more non-fatty acid anti-inflammatory drugs, and particularly, but not exclusively, to compositions and methods related to the production and use of non-methylene interrupted fatty acids such as sciadonic acids, juniperonic acid, pinoleic acid and dihomopinoleic acid in combination with one or more anti-inflammatory drugs.

### CROSS-REFERENCE TO RELATED APPLICATIONS

This application claims priority to and the benefit of U.S. Provisional Application No. 62/269,494, filed Dec. 18, 2015, which is hereby incorporated by reference in its entirety.

### FIELD OF TECHNOLOGY

Provided herein is technology relating to compositions containing bioactive fatty acids in combination with one or more non-fatty acid anti-inflammatory drugs, and particularly, but not exclusively, to compositions and methods related to the production and use of non-methylene interrupted fatty acids such as sciadonic acids, juniperonic acid, pinoleic acid and dihomopinoleic acid in combination with one or more anti-inflammatory drugs.

### BACKGROUND

Inflammation is associated with a number of diseases and conditions. However, a number of anti-inflammatory drugs are ineffective and have substantial side effects, especially when taken at increased dosages through frequent administration.

What is needed in the art are improved compounds, compositions and formulations that allow the dosage and frequency of administration of anti-inflammatory drugs to be reduced.

### SUMMARY

Provided herein is technology relating to compositions containing bioactive fatty acids in combination with one or more non-fatty acid anti-inflammatory drugs, and particularly, but not exclusively, to compositions and methods related to the production and use of non-methylene interrupted fatty acids such as sciadonic acids, juniperonic acid, pinoleic acid and dihomopinoleic acid in combination with one or more anti-inflammatory drugs.

In some embodiments, the present invention provides methods of reducing or treating inflammation in a subject in thereof comprising co-administering a lipid composition comprising fatty acids having a non-methylene interrupted bond system and non-fatty acid anti-inflammatory agent.

In other embodiments, the present invention provides for the use of a combination of a lipid composition comprising fatty acids having a non-methylene interrupted bond system and a non-fatty acid anti-inflammatory agent to reduce or treat inflammation in a subject in need thereof.

In other embodiments, the present invention provides formulations comprising effective amounts of a lipid composition comprising fatty acids having a non-methylene interrupted bond system and a non-fatty acid anti-inflammatory agent, wherein said effective amounts are sufficient to reduce or alleviate inflammation in a subject in need thereof.

In some embodiments, the lipid composition comprising fatty acids having a non-methylene interrupted bond system comprises one of more of a non-methylene interrupted fatty acid selected from the group consisting of sciadonic acid, juniperonic acid, pinoleic acid, dihomopinoleic acid, taxoleic acid (5,9 18:2, coniferonic acid (5,9,12,15 18:4), 5,11 18:2 fatty acid, 5,11 20:2 fatty acid; 5,13 20:2 fatty acid, 7,13 22:2 fatty acid, 7,15 22:2 fatty acid and synthetic fatty acids selected from the group consisting of 1, 11, 14, 17 20:4; 2, 11, 14, 17 20:4; 3, 11, 14 17 20:4; 4, 11, 14 17 20:4; 6, 11,14 17 20:4; 7,11,14 17 20:4; 1, 9, 12, 15 18:4; 2, 9, 12, 15 18:4; 3, 9, 12, 15 18:4; 4, 9, 12, 15 18:4, 5, 9, 12, 15 18:4, 1, 11, 14 20:3; 2, 11, 14 20:3; 3, 11, 14 20:3; 4, 11, 14 20:3; 6, 11,14 20:3; 1, 9, 12 18:3; 2, 9, 12 18:3; 3, 9, 12 18:3; and 4, 9, 12 18:3 fatty acids. In some embodiments, the lipid composition comprising fatty acids having a non-methylene interrupted bond system comprises greater than about 5% w/w of said fatty acids, greater than about 10% w/w of said fatty acids, greater than about 15% w/w of said fatty acids, or greater than about 20% w/w of said fatty acids. In some embodiments, the lipid composition comprising fatty acids having a non-methylene interrupted bond system comprises lipids comprising said fatty acids in a form selected from the group consisting of free fatty acids, ethyl esters, triglycerides, phospholipids and combinations thereof.

In some embodiments, the non-fatty acid anti-inflammatory agent is selected from the group consisting of a steroidal anti-inflammatory agent, a small molecule drug anti-inflammatory agent, and a biologic anti-inflammatory agent. In some embodiments, the steroidal anti-inflammatory agent is hydrocortisone. In some embodiments, the anti-inflammatory agent is a small molecule drug. In some embodiments, the small molecule drug is a non-steroidal anti-inflammatory drug. In some embodiments, the non-steroidal anti-inflammatory drug is selected from the group consisting of salicylates, propionic acid derivatives, acetic acid derivatives, fenamic acid derivatives, biphenylcarboxylic acid derivatives and oxicams. In some embodiments, the non-steroidal anti-inflammatory drug is selected from the group consisting of acetylsalicylic acid), diflunisal (Dolobid), salicylic acid, salsalate (Disalcid), Ibuprofen, Dexibuprofen, Naproxen, Fenoprofen, Ketoprofen, Dexketoprofen, Flurbiprofen, Oxaprozin, Loxoprofen, Indomethacin, Tolmetin, Sulindac, Etodolac, Ketorolac, Diclofenac, Aceclofenac, Nabumetone, Piroxicam, Meloxicam, Tenoxicam, Droxicam, Lornoxicam, Isoxicam, Phenylbutazone, Mefenamic acid, Meclofenamic acid, Flufenamic acid, Tolfenamic acid, Celecoxib, Rofecoxib, Valdecoxib, Parecoxib, Lumiracoxib, Etoricoxib, Firocoxib. Nimesulide. Clonixin, Licofelone, and H-harpagide. In some embodiments, the biologic anti-inflammatory agent is selected from the group consisting of Abrilumab, Adalimumab, ALD518, Atlizumab, Brodalumab, Canakinumab, Certolizumab pegol, Clazakizumab, Clenoliximab, Efalizumab, Eldelumab, Erlizumab, Etrolizumab, Fasinumab, Fezakinumab, Fletikumab, Fontolizumab, Golimumab, Guselkumab, Infliximab, Ixekizumab, Lulizumab pegol, Mavrilimumab, Natalizumab, Ocrelizumab, Ozoralizumab, Perakizumab, Priliximab, Reslizumab, Rontalizumab, Ruplizumab, Setoxaximab, Sifalimumab, Siplizumab, Sirukumab, Talizumab, Tildrakizumab, Tocilizumab, Ustekinumab, Vedolizumab, Vepalimomab, Visilizumab, Zanolimumab, Zolimomab aritox.

In some embodiments, the inflammation is caused by or associated with a disease or condition selected from the group consisting of restenosis, arteriosclerosis, coronary heart disease, thrombosis, myocardial infarction, stroke, hypertension, fatty liver, diabetes, hyperglycaemia, hyperinsulinemia, and stenosis, rheumatoid arthritis, systemic vasculitis, systemic lupus erythematosus, systemic sclerosis, dermatomyositis, polymyositis, various autoimmune endocrine disorders (e.g. thyroiditis and adrenalitis), inflammatory bowel diseases and colitis (e.g., Crohn's colitis), nephritis, various inflammatory skin disorders (e.g. psoriasis, atopic dermatitis and food allergy) and acute and chronic allograft rejection after organ transplantation. In some embodiments, the agents are co-administered under conditions such that the disease or condition is alleviated or improved as compared to an untreated state. In some embodiments, the agents are co-administered under conditions such that the dosage and/or frequency of administration of the non-fatty anti-inflammatory agent can be reduced.

Additional embodiments will be apparent to persons skilled in the relevant art based on the teachings contained herein.

### DETAILED DESCRIPTION

Provided herein is technology relating to compositions containing bioactive fatty acids in combination with one or more non-fatty acid anti-inflammatory drugs, and particularly, but not exclusively, to compositions and methods related to the production and use of non-methylene interrupted fatty acids such as sciadonic acids, juniperonic acid, pinoleic acid and dihomopinoleic acid in combination with one or more anti-inflammatory drugs.

This technology is described below, wherein the section headings are for organizational purposes only and are not to be construed as limiting the described subject matter in any way.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the technology may be readily combined, without departing from the scope or spirit of the technology.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, “feeding” refers to providing a substance, compound, composition, etc. to a living organism. For example, the substance, compound, composition, etc. may be an energy source, a carbon source, a nutrient, or a source of other elements, molecules, and/or precursors of biological molecules that are used by the living organism and/or are metabolized (e.g., catabolized, anabolized) by the living organism. The substance, compound, composition, etc. is not necessarily a substance, compound, composition, etc. that the living organism encounters in its native milieu, but may be a synthetic substance, compound, composition, etc. or a natural substance, compound, composition, etc. that is nevertheless used by the living organism for metabolism. The substance, compound, composition, etc. may be added to a culture medium or a substrate in which or on which the living organism lives and/or grows.

As used herein, “active” or “activity” refers to native or naturally occurring biological and/or immunological activity.

As used herein the term, “in vitro” refers to an artificial environment and to processes or reactions that occur within an artificial environment. In vitro environments may include, but are not limited to, test tubes and cell cultures. The term “in vivo” refers to the natural environment (e.g., an animal or a cell) and to processes or reactions that occur within a natural environment.

As used herein, the terms “subject” and “patient” refer to any animal, such as a mammal like a dog, cat, bird, livestock, and preferably a human (e.g., a human with a disease such as obesity, diabetes, or insulin resistance).

As used herein, the term “individual” refers to vertebrates, particularly members of the mammalian species. The term includes but is not limited to domestic animals, sports animals, primates, and humans.

As used herein, the term “effective amount” refers to the amount of a composition sufficient to effect beneficial or desired results. An effective amount can be administered in one or more administrations, applications, or dosages and is not intended to be limited to a particular formulation or administration route.

As used herein, the term “administration” refers to the act of giving a drug, prodrug, or other agent, or therapeutic treatment to a subject. Exemplary routes of administration to the human body can be through the eyes (ophthalmic), mouth (oral), skin (transdermal, topical), nose (nasal), lungs (inhalant), oral mucosa (buccal), ear, by injection (e.g., intravenously, subcutaneously, intratumorally, intraperitoneally, etc.), and the like.

As used herein, the term “co-administration” refers to the administration of at least two agents or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent.

As used herein, the term “pharmaceutical composition” refers to the combination of an active agent with a carrier, inert or active, making the composition especially suitable for therapeutic use.

The terms “pharmaceutically acceptable” or “pharmacologically acceptable”, as used herein, refer to compositions that do not substantially produce adverse reactions, e.g., toxic, allergic, or immunological reactions, when administered to a subject.

As used herein, the term “treating” includes reducing or alleviating at least one adverse effect or symptom of a disease or disorder through introducing in any way a therapeutic composition of the present technology into or onto the body of a subject. “Treatment” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present technology.

### Embodiments of the Technology

Provided herein is technology relating to compositions containing bioactive fatty acids in combination with one or more non-fatty acid anti-inflammatory drugs, and particularly, but not exclusively, to compositions and methods related to the production and use of non-methylene interrupted fatty acids such as sciadonic acids, juniperonic acid, pinoleic acid and dihomopinoleic acid in combination with one or more anti-inflammatory drugs. Below, sources of bioactive fatty acids and lipids, lipid compositions comprising bioactive fatty acids, methods for making the compositions and uses of the compositions are described. The present invention contemplates that the combined administration of the bioactive fatty acids and one or more anti-inflammatory drugs provides a synergistic action in reducing inflammation and in treating diseases associated with inflammation.

### Non-Methylene-Interrupted Fatty Acids

The term non-methylene-interrupted fatty acid, the acronym for which is NMIFA, refers to a fatty acid with a series of double bonds in which at least one adjacent pair of double bonds is separated by at least two carbon atoms, i.e., by a group other than a single methylene group. Examples of NMIFA include, but are not limited to, 5,11,14-eicosatrienoic acid (sciadonic acid); 5,9,12-cis-octadecatrienoic acid (pinolenic acid); and 5,11,14,17-eicosatetraenoic acid (juniperonic acid). Preferred NMIFAs have the following formula, wherein the NMIFA is an acid, a salt or an ester, and R1 is a C₁-C₅ alkyl group and R2 is a C₂-C₆alkyl group, may be advantageously used for the preparation of a composition intended to modulate the metabolism of lipids in superficial mammalian tissues.

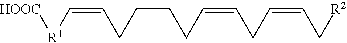

Particularly preferred NMIFAs are those in which R1 is a C₃ alkyl group and R2 is a C₂-C₆ alkyl group, or in which R2 is a C₄ alkyl group and R1 is a C₁-C₅ alkyl group. The most preferred is that in which R1 is an n-propyl group and R2 is an n-butyl group (5,11,14-eicosatrienoic acid, also called 20:3(5,11,14)). The NMIFAs may be preferably provided as triglycerides, phospholipids, fatty acids ester, free fatty acids or combinations thereof.

Sciadonic acid (5,11,14-eicosatrienoic acid, 20:3Δ5,11,14) is a polyunsaturated fatty acid containing non-methylene-interrupted double bonds, such as a Δ5-ethylenic bond. Sciadonic acid is often found in gymnosperms, in seed oils, leaves, and wood. It is also found in a few angiosperms, especially in seed oils. Sciadonic acid has several biological activities, including lowering triglyceride and cholesterol levels, reducing reperfusion injury, modifying autoimmune response, having cannabimimetic effect, treatment of skin disease, and treatment of sensitive or dry skin. WO 95/17987 (The Regents of the University of California) shows that broad class of NMIFAs, including 5,11,14-eicosatrienoic acid, may be used in an effective amount for suppressing autoimmune diseases in general, for example rheumatoid arthritis, lupus erythmatosis, multiple sclerosis, myasthenia gravis, and about 30 other diseases currently known. NMIFAs, including 5,11,14-eicosatrienoic acid, are further described in U.S. Pat. Nos. 5,456,912 and 6,280,755 as well as US Publ. No. 20120156171, each of which is incorporated herein by reference in its entirety.

Pinolenic acid ((5Z,9Z,12Z)-octadeca-5,9,12-trienoic acid; all-cis-5,9,12-18:3) is a fatty acid contained in Siberian Pine nuts, Korean Pine nuts and the seeds of other pines (Pinus species). The highest percentage of pinolenic acid is found in Siberian pine nuts and the oil produced from them. JP 61 058 536 (Nippon Oil) discloses a method for purifying pine nut oil containing at least 10% by weight of 5,9,12-cis-octadecatrienoic acid which exhibits a curative effect against arterial hypertension. WO 96 05 164 (Broadben Nominees Pty) discloses an anti-inflammatory preparation comprising a purified active fraction, for example 5,11,14,17-eicosatetraenoic acid, isolated from a lipid extract of Perna canalicullus or Mytilus edulis. Dihomopinoleic acid also finds use in the compositions of the present invention.

Some of the NMIFAs of the invention are naturally occurring substances. Others may be synthesized according to well-known published methodology (see for example Evans et al., Chem. Phys. Lipids, 38, 327-342, 1995).

For example, 20:3(5,11,14) is a naturally occurring substance which generally occurs as one fatty acid in a mixture of fatty acids. This NMIFA is found in a wide variety of plants as minor or major fraction of the total fatty acid composition. Both the extraction of the mixture of fatty acid from their natural sources and the extraction of the 20:3(5,11,14) from the resulting fatty acids can be achieved by conventional extraction and purification methods well known among those skilled in the art.

The natural sources of fatty acids containing 20:3(5,11,14) are primarily plant seeds, and prominent among these are conifers and ornamental shrubs. The seed oils from these plants are similar to normal edible oils, containing largely oleic, linoleic and linolenic acids, but also containing useful amounts of NMIFAs. Table 1 lists examples of seeds whose lipid contents contain significant amounts of 20:3(5,11,14).

| | % of | | % of |
|---|---|---|---|
| | 20:3 (5, 11, 14) | | 20:3 (5, 11, 14) |
| | among total | | among total |
| Source | fatty acids | Source | fatty acids |
| Juniperis | 14.8 | Sciadopitys | 15 |
| virginiensis | | verticallata | |
| Plarycladus | 3 | Caltha | 23 |
| orientalis | | palustris | |
| Juniperis | 12.3 | Calitrus | 14 |
| chinesis | | rhombaidea | |
| Torreya | 7 | Mortierella | 7 |
| nucifera | | alpina* | |
| Podocarpus | 24 | Ephedra | 22 |
| nagi | | campylopoda | |
| Anemone | 10 | Anemone | 6 |
| rivularis | | leveillei | |
| Cimaifuga | 6 | Erantis | 6 |
| racemosa | | hyemalis | |
| Gingko | 2.2 | Pinus | 7 |
| biloba | | silvestris | |

*see the Japanese patent JP5276964 (Suntory LTD)

Purification of 20:3(5,11,14) may be in particular achieved by (1) choosing a starting seed source high in total fat content and 20:3(5,11,14) content but not containing other contaminating trienes, in particular alpha-linolenic acid (18:3n-3) and gamma-linolenic acid (18:3n-6) (Podocarpus nagi, Table 1, is such an example); (2) extracting the lipids with isopropanol and chloroform according to the method of Nichols (Biochim. Biophys Acta 70: 417, 1963); (3) conventional degumming and decoloring methods; (4) preparing methyl esters with 2% methanolic sulfuric acid according to the method of Christie (p. 52-53, in Lipid Analysis, Pergamon Press, Oxford, 1982); (5) eluting 20:3(5,11,14) methyl ester from a silver nitrate impregnated acid-washed Florisil column with a hexane:ether mixture ranging from 9:1 to 8:2 (volume/volume) according to Carroll, J. Am. Oil Chem. Soc. 40: 413, 1963; Wilner, Chem. Ind (Lond) Oct., 30: 1839, 1965; Merck ChromNews 4(1): 1995; Anderson, J. Lipid Res. 6: 577, 1965; and Teshima, Bull. Jap. Soc. Scien. Fish. 44: 927, 1978); (6) removing contaminating silver ions by the method of Akesson (Eur. J. Biochem. 9:463, 1969); and (7) optionally converting the methyl ester back to the free acid form by saponification in 1 M potassium hydroxide in 95% ethanol according to Christie (p. 51-52, in Lipid Analysis, Pergamon Press, Oxford, 1982).

20:4 fatty acids and analogs are utilized in the present invention. In referred embodiments, the 20:4 fatty acids have a non-methylene-interrupted bond system. Natural sources of 20:4 fatty acids and analogs, especially juniperonic acid (5, 11, 14, 17 20:4) include juniper berries from various Juniperus spp., including, but not limited to: J. communis, J. chinensis, J. conferta , J. rigida , J. brevifolia, J. cedrus , J. deltoides , J. formosana , J. lutchuensis , J. navicularis, J. oxycedrus , J. macrocarpa , J. drupacea , J. convallium , J. excels, J. foetidissima, J. indica, J. komarovii , J. phoenicea, J. pingii, J. procera, J. procumbens, J. pseudosabina , J. recurve, J. sabina, J. saltuaria, J. semiglobosa, J. squamata, J. thurifera, J. fibefica, J. wallichiana, J. angosturana, J. ashei , J. arizonica , J. barbadensis, J. bermudiana, J. blancoi, J. californica, J. coahuilensis, J. comitana, J. deppeana , J. durangensis , J. flaccida , J. gamboana , J. horizontalis, J. jaliscana, J. monosperma , J. monficola, J. occidentalis , J. osteosperma , J. pinchotii, J. saltillensis , J. scopulorum, J. standleyi and J. virginiana.

In some preferred embodiments, an oil (juniper berry oil) is extracted from juniper berry powder, most preferably a powder made from J. communis or J. chinensis berries. In some especially preferred embodiments, the juniper berry oil is extracted from the waste or residue from juniper berry oil processing for essential oils, i.e., juniper berries that have been previously subjected to steam distillation, or from juniper berries that have been used in the production of gin or other flavored spirits. For sources of juniper berry waste or residues, see, e.g., Slankovic, M. and Randjelovic, M. Hem. Ind. 1980, 34, 355-377; Dyr, J. and Uher, J. Czech. Pat. 134,003, 1970; Grog, I. Kern. Ind. 1983, 32, 575-578; and Veljkovic et al., Enz. Microb. Technol. 1990, 12, 706-709.

Juniper berries have been previously extracted in for the essential oil market by steam distillation or soaking the berries in vegetable. This extracts aromatic components, but not triglycerides or other fatty acid containing components. In contrast, in preferred embodiments of the present invention, juniper oil comprising or consisting essentially of triglycerides is extracted a juniper feedstock (e.g., juniper berries, juniper berry powder, steam-distilled juniper berries or juniper berry waste (e.g., juniper berry residue from gin or other spirit manufacturing). In some embodiments, the outer coat of the juniper berries in the juniper feedstock is removed by treatment with acid. In some embodiments, oil is extracted from the juniper feedstock by cold pressing. In other embodiments, the oil is extracted from the juniper feedstock by solvent extraction. Suitable solvents include food grade solvents such as n-hexane and cyclohexane, liquid propane, isopropanol, acetone, ethyl acetate, ethanol and combination thereof. In other embodiments, super critical fluid extraction using carbon dioxide is utilized to extract oil from the juniper feedstock. Juniper oils can contain certain irritants, hence in some embodiments, conventional processing (washing, bleaching, deodorizing) are employed.

In some preferred embodiments, the extracted oil comprises or consists essentially of triglycerides. The oil obtained from the extraction is generally characterized as having a ratio of juniperonic acid to sciadonic acid of from about 1:5 to 5:1, 1:2.5 to 2.5:1, 1:2 to 2:1, 1:1.5 to 1.5:1, 1:1 to 3:1, 1:5 to 2.5:1, 1:3 to 1:1 or 1:1 to 1:1.5. In some embodiments, the weight percent of juniperonic acid expressed as grams per 100 grams fatty acids in the oil is from about 5% to 20%, 5% to 15%, 8% to 15%, or 3% to 10%. In some embodiments, the weight percent of sciadonic acid expressed as grams per 100 grams fatty acids in the oil is from about 5% to 20%, 5% to 15%, 8% to 20%, or 3% to 10%.

Purification of juniperonic acid may be in particular achieved by (1) choosing a starting juniper feedstock as described above (2) extracting the lipids with a suitable solvent as described above; (3) conventional degumming and decoloring methods; (4) preparing ethyl esters; (5) separating the juniperonic acid by molecular distillation of the ethyl esters or lower molecular weight, and (7) optionally converting the ethyl ester back to the free acid form.

In some embodiments, the juniper oil is formulated for oral delivery, for example by encapsulation in a soft gel capsule or as described in more detail below. In some embodiments, the juniper oil is protected from oxidation by formulating the juniper oil with an antioxidant. Suitable antioxidants include, but are not limited to, ascorbic acid and tocopherol.

### Non-Fatty Acid Anti-Inflammatory Drugs

The present invention contemplates that bioactive fatty acids described above will be co-administered with one or more non-fatty acid anti-inflammatory drugs to an individual in need thereof. Non-fatty acid anti-inflammatory drugs are defined herein as small molecule drugs or antibodies (including humanized antibodies, single chain antibodies and antibody fragments) that have an anti-inflammatory effect in animals or humans. This definition specifically excludes anti-inflammatory fatty acids such as EPA, DHA and DPA.

Examples of small organic compounds useful in the present invention include, but are not limited to, non-steroidal anti-inflammatory drugs (NSAIDS)(the NSAIDS can, for example, be selected from the following categories: (e.g., salicylates, propionic acid derivatives, acetic acid derivatives, fenamic acid derivatives, biphenylcarboxylic acid derivatives and oxicams)). Examples of silicylates include, but are not limited to, aspirin (acetylsalicylic acid), diflunisal (Dolobid), salicylic acid, and salsalate (Disalcid). Examples of propionic acid derivatives include, but are not limited to, Ibuprofen, Dexibuprofen, Naproxen, Fenoprofen, Ketoprofen, Dexketoprofen, Flurbiprofen, Oxaprozin and Loxoprofen. Examples of acetic acid derivatives include, but are not limited to, Indomethacin, Tolmetin, Sulindac, Etodolac, Ketorolac, Diclofenac, Aceclofenac, and Nabumetone. Examples of enolic acid (Oxicam) derivatives include, but are not limited to, Piroxicam, Meloxicam, Tenoxicam, Droxicam, Lornoxicam, Isoxicam, and Phenylbutazone. Examples of anthranilic acid derivatives (Fenamates) include, but are not limited to, Mefenamic acid, Meclofenamic acid, Flufenamic acid, and Tolfenamic acid. Examples of selective COX-2 inhibitors (Coxibs)include, but are not limited to, Celecoxib, Rofecoxib, Valdecoxib, Parecoxib, Lumiracoxib, Etoricoxib and Firocoxib. Example of Sulfonanilides include, but are not limited to, Nimesulide. Other small small molecule anti-inflammatories include, but are not limited to, Clonixin, Licofelone, and H-harpagide.

Examples of steroidal anti-inflammatory drugs useful in the present invention include, but are not limited to, hydrocortisone and the like.

Examples of biologic drugs (e.g., monoclonal antibodies, single chain antibodies, antibody fragments, and the like) useful in the present invention include, but are not limited to Abrilumab, Adalimumab, ALD518, Atlizumab, Brodalumab, Canakinumab, Certolizumab pegol, Clazakizumab, Clenoliximab, Efalizumab, Eldelumab, Erlizumab, Etrolizumab, Fasinumab, Fezakinumab, Fletikumab, Fontolizumab, Golimumab, Guselkumab, Infliximab, Ixekizumab, Lulizumab pegol, Mavrilimumab, Natalizumab, Ocrelizumab, Ozoralizumab, Perakizumab, Priliximab, Reslizumab, Rontalizumab, Ruplizumab, Setoxaximab, Sifalimumab, Siplizumab, Sirukumab, Talizumab, Tildrakizumab, Tocilizumab, Ustekinumab, Vedolizumab, Vepalimomab, Visilizumab, Zanolimumab, Zolimomab aritox.

### Bioactive Lipid Formulations

The present invention provides bioactive lipid formulations comprising one or more bioactive fatty acids, and in particularly preferred embodiments NMIFAs, in combination with a non-fatty acid anti-inflammatory drug. In some embodiments, the bioactive fatty acid and non-fatty acid anti-inflammatory drug are formulated in the same delivery vehicle (e.g., a soft gel capsule) while in other embodiments, the bioactive fatty acid non-fatty acid anti-inflammatory drug are provided in separate delivery vehicles.

It will be understood that the fatty acids may be provided in the formulation as free fatty acids, as ethyl esters, or in the form of diglycerides, triglycerides, or phospholipids to which the fatty acid is attached. The bioactive lipid formulations are preferably characterized by comprising a particular ratio of the bioactive fatty acids to one another or as having a defined weight/weight (w/w) percentage of the bioactive fatty acids which refers to the weight of the specific fatty acid per total weight of fatty acids in the formulation (i.e, grams the specified acid per 100 grams of fatty acids in the lipid formulation).

Thus, the lipid formulations according to the present technology are either fatty acids analogous to naturally occurring fatty acids, especially NMIFAs or their analogs, alone in combination with other bioactive fatty acids, or naturally occurring lipids comprising the fatty acids. Incorporation of the fatty acids in naturally occurring lipids (e.g., monoglycerides, diglycerides, triglycerides, and/or phospholipids) produces a compound with different absorption characteristics compared to free fatty acids. In addition, it is contemplated that incorporating fatty acids in naturally occurring lipids (e.g., monoglycerides, diglycerides, triglycerides, and/or phospholipids) may also increase the bioavailability or stability.

In some embodiments, the fatty acids in the lipid formulation are esterified to a triglyceride, diglyceride, monoglyceride or phospholipid molecule. In some embodiments, the fatty acids in the lipid formulation are provided as ethyl esters.

In some embodiments, the fatty acids in the lipid formulations are provided by blending one or more oils or lipids. In some embodiments, the fatty acids are provided by pine nut oil, juniper oil, and other natural sources as described above.

In some embodiments, the lipid formulations are suitable for human consumption on a daily basis for an extended period of time, e.g., 1 month, 2 months, 6 months, 1 year or 2 years, when provided in daily dosage of from 200 mg to 5 or 10 grams. In some embodiments, the lipid formulations further comprise a food safe antioxidant. In some embodiments, the lipid formulations are provided in an oral delivery vehicle, food product, nutritional supplement, dietary supplement or functional food.

The present invention likewise provides methods of using the lipid formulations. These methods and uses are described in detail below but may be summarized as follows. In some embodiments, the present invention provides methods of treating a subject suffering from inflammation comprising administering to the subject the bioactive lipid and non-fatty acid ant-inflammatory drug formulation/s or oral delivery vehicle/s, food product, nutritional supplement, dietary supplement or functional food comprising the lipid formulation to a subject in need thereof. In some embodiments, the administration is oral, topical, parenteral, enteral, transdermal, intradermal, intraocular, intravitreal, sublingual, or intravaginal and may preferably comprise an effective amount of the composition.

In further preferred embodiments, the present invention provides methods of reducing inflammation and/or alleviating or improving one or more of the following diseases or conditions: restenosis, arteriosclerosis, coronary heart disease, thrombosis, myocardial infarction, stroke, hypertension, fatty liver, diabetes, hyperglycaemia, hyperinsulinemia, and stenosis, rheumatoid arthritis, systemic vasculitis, systemic lupus erythematosus, systemic sclerosis, dermatomyositis, polymyositis, various autoimmune endocrine disorders (e.g. thyroiditis and adrenalitis), inflammatory bowel diseases and colitis (e.g., Crohn's colitis), nephritis, various inflammatory skin disorders (e.g. psoriasis, atopic dermatitis and food allergy) and acute and chronic allograft rejection after organ transplantation, comprising administering to a subject in need thereof the formulation, food product, nutritional supplement, dietary supplement or function food as described above. In some embodiments, the administration or oral, topical, parenteral, enteral, transdermal, intradermal, intraocular, intravitreal, sublingual, or intravaginal and may preferably comprise an effective amount of the composition. The treatment is preferably performed under conditions such that the disease or condition is alleviated or improved as compared to an untreated state.

Provided herein are pharmaceutical compositions comprising a therapeutically effective amount of a composition according to the present technology and a pharmaceutically acceptable carrier, diluent, or excipient (including combinations thereof).

A composition according to the technology comprises or consists of a therapeutically effective amount of a pharmaceutically active agent. In some embodiments, it includes a pharmaceutically acceptable carrier, diluent, or excipient (including combinations thereof). Acceptable carriers or diluents for therapeutic use are well known in the pharmaceutical art and are described, for example, in Remington's Pharmaceutical Sciences, Mack Publishing Co. (A. R. Gennaro edit. 1985). The choice of pharmaceutical carrier, excipient, or diluent is selected with regard to the intended route of administration and standard pharmaceutical practice. The pharmaceutical comprise as, or in addition to, the carrier, excipient, or diluent any suitable binder(s), lubricant(s), suspending agent(s), coating agent(s), and/or solubilizing agent(s).

This pharmaceutical composition will desirably be provided in a sterile form. It may be provided in unit dosage form and will generally be provided in a sealed container. A plurality of unit dosage forms may be provided.

Pharmaceutical compositions within the scope of the present technology may include one or more of the following: preserving agents, solubilizing agents, stabilizing agents, wetting agents, emulsifiers, sweeteners, colorants, flavoring agents, odorants, and/or salts. Compounds of the present technology may themselves be provided in the form of a pharmaceutically acceptable salt. In addition, embodiments may comprise buffers, coating agents, antioxidants, suspending agents, adjuvants, excipients, and/or diluents. Examples of preservatives include sodium benzoate, sorbic acid, and esters of p-hydroxybenzoic acid.

They may also contain other therapeutically active agents in addition to compounds of the present technology. Where two or more therapeutic agents are used they may be administered separately (e.g., at different times and/or via different routes) and therefore do not always need to be present in a single composition. Thus, combination therapy is within the scope of the present technology.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, via the penis, vaginal, epidural, sublingual.

It is to be understood that not all of the agent need be administered by the same route. Likewise, if the composition comprises more than one active component, then those components may be administered by different routes.

If the agent of the present technology is administered parenterally, then examples of such administration include one or more of: intravenously, intra-arterially, intraperitoneally, intrathecally, intraventricularly, intraurethrally, intrastemally, intracranially, intramuscularly, or subcutaneously administering the agent; and/or by using infusion techniques.

In some embodiments, pharmaceutical compositions adapted for oral administration are provided as capsules or tablets; as powders or granules; as solutions, syrups or suspensions (in aqueous or non-aqueous liquids); as edible foams or whips; or as emulsions. Tablets or hard gelatine capsules may comprise lactose, maize starch or derivatives thereof, stearic acid or salts thereof. Soft gelatine capsules may comprise vegetable oils, waxes, fats, semi-solid, or liquid polyols etc. Solutions and syrups may comprise water, polyols and sugars. For the preparation of suspensions, oils (e.g., vegetable oils) may be used to provide oil-in-water or water-in-oil suspensions. An active agent intended for oral administration may be coated with or admixed with a material that delays disintegration and/or absorption of the active agent in the gastrointestinal tract (e.g., glyceryl monostearate or glyceryl distearate may be used). Thus, the sustained release of an active agent may be achieved over many hours and, if necessary, the active agent can be protected from being degraded within the stomach. Pharmaceutical compositions for oral administration may be formulated to facilitate release of an active agent at a particular gastrointestinal location due to specific pH or enzymatic conditions.

Alternatively, the agent of the present technology can be administered in the form of a suppository or pessary, or it may be applied topically in the form of a gel, hydrogel, lotion, solution, cream, ointment or dusting powder. The agent of the present technology may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the agent of the present technology can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

If the agent of the present technology is administered parenterally, then examples of such administration include one or more of: intravenously, intra-arterially, intraperitoneally, intrathecally, intraventricularly, intraurethrally, intrasternally, intracranially, intramuscularly or subcutaneously administering the agent; and/or by using infusion techniques.

For parenteral administration, the agent is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Typically, a physician will determine the actual dosage which will be most suitable for an individual subject. The specific dose level and frequency of dosage for any particular patient may be varied and will depend upon a variety of factors including the activity of the specific compound employed; the metabolic stability and length of action of that compound; the age, body weight, general health, sex, diet, mode and time of administration; rate of excretion; drug combination; the severity of the particular condition; and the individual undergoing therapy. The agent and/or the pharmaceutical composition of the present technology may be administered in accordance with a regimen of from 1 to 10 times per day, such as once or twice per day. For oral and parenteral administration to human patients, the daily dosage level of the agent may be in single or divided doses.

Depending upon the need, the agent may be administered at a dose of from 0.01 to 30 mg/kg body weight, such as from 0.1 to 10 mg/kg or from 0.1 to 1 mg/kg body weight. Naturally, the dosages mentioned herein are exemplary of the average case. There can, of course, be individual instances where higher or lower dosage ranges are merited.

“Therapeutically effective amount” refers to the amount of the therapeutic agent that is effective to achieve its intended purpose, i.e., reduction of inflammation and associated symptoms. While individual patient needs may vary, determination of optimal ranges for effective amounts of the compounds related to the technology is within the skill of the art. Generally, the dosage regimen for treating a condition with the compounds and/or compositions of this technology is selected in accordance with a variety of factors, including the type, age, weight, sex, diet and medical condition of the patient; the severity of the dysfunction; the route of administration; pharmacological considerations such as the activity, efficacy, pharmacokinetic and toxicology profiles of the particular compound used; whether a drug delivery system is used; and whether the compound is administered as part of a drug combination and can be adjusted by one skilled in the art. Thus, the dosage regimen actually employed may vary widely and therefore may deviate from the exemplary dosage regimens set forth herein.

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the technology as claimed should not be unduly limited to such specific embodiments. Indeed, various modifications of the described modes for carrying out the technology that are obvious to those skilled in pharmacology, biochemistry, medical science, or related fields are intended to be within the scope of the following claims.

We claim:

1-32. (canceled)

33. A formulation comprising effective amounts of a lipid composition comprising fatty acids having a non-methylene interrupted bond system and a non-fatty acid anti-inflammatory agent that does not occur naturally with the fatty acids having a non-methylene interrupted bond system, wherein said effective amounts are sufficient to reduce or alleviate inflammation in a subject in need thereof

34. The formulation of claim 33, wherein said lipid composition comprising fatty acids having a non-methylene interrupted bond system comprises one of more of a non-methylene interrupted fatty acid selected from the group consisting of sciadonic acid, juniperonic acid, pinoleic acid, dihomopinoleic acid, taxoleic acid (5,9 18:2, coniferonic acid (5,9,12,15 18:4), 5,11 18:2 fatty acid, 5,11 20:2 fatty acid; 5,13 20:2 fatty acid, 7,13 22:2 fatty acid, 7,15 22:2 fatty acid and synthetic fatty acids selected from the group consisting of 1, 11, 14, 17 20:4; 2, 11, 14, 17 20:4; 3, 11, 14 17 20:4; 4, 11, 14 17 20:4; 6, 11,14 17 20:4; 7,11,14 17 20:4; 1, 9, 12, 15 18:4; 2, 9, 12, 15 18:4; 3, 9, 12, 15 18:4; 4, 9, 12, 15 18:4, 5, 9, 12, 15 18:4, 1, 11, 14 20:3; 2, 11, 14 20:3; 3, 11, 14 20:3; 4, 11, 14 20:3; 6, 11,14 20:3; 1, 9, 12 18:3; 2, 9, 12 18:3; 3, 9, 12 18:3; and 4, 9, 12 18:3 fatty acids.

35. The formulation of claim 33, wherein said lipid composition comprising fatty acids having a non-methylene interrupted bond system comprises greater than about 5% w/w of said fatty acids.

36. The formulation of claim 33, wherein said lipid composition comprising fatty acids having a non-methylene interrupted bond system comprises greater than about 10% w/w of said fatty acids.

37. The formulation of claim 33, wherein said lipid composition comprising fatty acids having a non-methylene interrupted bond system comprises greater than about 15% w/w of said fatty acids.

38. The formulation of claim 33, wherein said lipid composition comprising fatty acids having a non-methylene interrupted bond system comprises greater than about 20% w/w of said fatty acids.

39. The formulation of claim 33, wherein said lipid composition comprising fatty acids having a non-methylene interrupted bond system comprises lipids comprising said fatty acids in a form selected from the group consisting of free fatty acids, ethyl esters, triglycerides, phospholipids and combinations thereof.

40. The formulation of any of claim 33, wherein said non-fatty acid anti-inflammatory agent is selected from the group consisting of a steroidal anti-inflammatory agent, a small molecule drug anti-inflammatory agent, and a biologic anti-inflammatory agent.

41. The formulation of any of claim 33, wherein said steroidal anti-inflammatory agent is hydrocortisone.

42. The formulation of any of claim 33, wherein said small molecule drug anti-inflammatory agent.

43. The formulation of any of claim 33, wherein said small molecule drug is a non-steroidal anti-inflammatory drug.

44. The formulation of claim 43, wherein said non-steroidal anti-inflammatory drug is selected from the group consisting of salicylates, propionic acid derivatives, acetic acid derivatives, fenamic acid derivatives, biphenylcarboxylic acid derivatives and oxicams.

45. The formulation of claim 43, wherein said non-steroidal anti-inflammatory drug is selected from the group consisting of acetylsalicylic acid), diflunisal (Dolobid), salicylic acid, salsalate (Disalcid), Ibuprofen, Dexibuprofen, Naproxen, Fenoprofen, Ketoprofen, Dexketoprofen, Flurbiprofen, Oxaprozin, Loxoprofen, Indomethacin, Tolmetin, Sulindac, Etodolac, Ketorolac, Diclofenac, Aceclofenac, Nabumetone, Piroxicam, Meloxicam, Tenoxicam, Droxicam, Lornoxicam, Isoxicam, Phenylbutazone, Mefenamic acid, Meclofenamic acid, Flufenamic acid, Tolfenamic acid, Celecoxib, Rofecoxib, Valdecoxib, Parecoxib, Lumiracoxib, Etoricoxib, Firocoxib. Nimesulide. Clonixin, Licofelone, and H-harpagide.

46. The formulation of claim 33, wherein said biologic anti-inflammatory agent is selected from the group consisting of Abrilumab, Adalimumab, ALD518, Atlizumab, Brodalumab, Canakinumab, Certolizumab pegol, Clazakizumab, Clenoliximab, Efalizumab, Eldelumab, Erlizumab, Etrolizumab, Fasinumab, Fezakinumab, Fletikumab, Fontolizumab, Golimumab, Guselkumab, Infliximab, Ixekizumab, Lulizumab pegol, Mavrilimumab, Natalizumab, Ocrelizumab, Ozoralizumab, Perakizumab, Priliximab, Reslizumab, Rontalizumab, Ruplizumab, Setoxaximab, Sifalimumab, Siplizumab, Sirukumab, Talizumab, Tildrakizumab, Tocilizumab, Ustekinumab, Vedolizumab, Vepalimomab, Visilizumab, Zanolimumab, Zolimomab aritox.

47. A method of reducing or treating inflammation in a subject in thereof comprising co-administering the formulation of claim 33.

48. The method claim 47, wherein said inflammation is caused by or associated with a disease or condition selected from the group consisting of restenosis, arteriosclerosis, coronary heart disease, thrombosis, myocardial infarction, stroke, hypertension, fatty liver, diabetes, hyperglycaemia, hyperinsulinemia, and stenosis, rheumatoid arthritis, systemic vasculitis, systemic lupus erythematosus, systemic sclerosis, dermatomyositis, polymyositis, various autoimmune endocrine disorders (e.g. thyroiditis and adrenalitis), inflammatory bowel diseases and colitis (e.g., Crohn's colitis), nephritis, various inflammatory skin disorders (e.g. psoriasis, atopic dermatitis and food allergy) and acute and chronic allograft rejection after organ transplantation.

49. The method of claim 48, wherein the formulation is administered under conditions such that the disease or condition is alleviated or improved as compared to an untreated state.
